(19) United States
(12) Patent Application Publication
(10) Pub. No.: US 2021/0282837 A1
(43) Pub. Date: Sep. 16, 2021

(54) THERMAL CUTTING-ASSISTED TISSUE RESECTION DEVICES AND SYSTEMS

(71) Applicant: Covidien LP, Mansfield, MA (US)

(72) Inventors: Kenneth E. Netzel, Loveland, CO (US); Purvishkumar H. Soni, Longmont, CO (US); Kenlyn S. Bonn, Lakewood, CO (US); Daniel A. Joseph, Golden, CO (US); John A. Hammerland, III, Arvada, CO (US); Daniel W. Mercier, Erie, CO (US); Devon E. Scott-Drechsel, Superior, CO (US); Mark A. Johnston, Boulder, CO (US); David M. Garrison, Longmont, CO (US); Jing Zhao, Superior, CO (US); Yanzhu Zhao, Blaine, MN (US)

(21) Appl. No.: 17/174,025

(22) Filed: Feb. 11, 2021

Related U.S. Application Data
(60) Provisional application No. 62/988,120, filed on Mar. 11, 2020.

(51) Int. Cl. A61B 18/14

(52) U.S. Cl. CPC A61B 18/149; A61B 2018/0091

(57) ABSTRACT

A tissue resection device includes a handpiece and an end effector assembly extending distally from the handpiece. The end effector assembly includes an outer shaft defining a longitudinal axis and including a window defined therethrough towards a distal end thereof. The end effector assembly further includes an inner member rotationally disposed within the outer shaft. The inner member includes a proximal body portion and a distal cutting portion. The proximal body portion is coaxially disposed on the longitudinal axis and rotatable thereabout. The distal cutting portion includes an offset portion that is radially offset from the longitudinal axis and is configured to orbit about the longitudinal axis. At least a portion of the distal cutting portion is a thermal cutting element configured to heat in response to application of electrical energy thereto.

### CROSS-REFERENCE TO RELATED APPLICATIONS

This application claims the benefit of, and priority to, U.S. Provisional Patent Application No. 62/988,120, filed on Mar. 11, 2020, the entire contents of which are hereby incorporated herein by reference.

### BACKGROUND

### Technical Field

The present disclosure relates generally to surgical devices and systems and, more particularly, to energy-based tissue resection devices and systems.

### Background of Related Art

Tissue resection may be performed endoscopically within an organ, such as a uterus, by inserting an endoscope (or hysteroscope) into the uterus and passing a tissue resection device through the endoscope (or hysteroscope) and into the uterus. With respect to such endoscopic tissue resection procedures, it often is desirable to distend the uterus with a fluid, for example, saline, sorbitol, or glycine. The inflow and outflow of the fluid during the procedure maintains the uterus in a distended state and flushes tissue and other debris from within the uterus to maintain a visible working space. Tissue resection may also be performed in open and/or other surgical procedures.

### SUMMARY

As used herein, the term “distal” refers to the portion that is described which is farther from an operator (whether a human surgeon or a surgical robot), while the term “proximal” refers to the portion that is being described which is closer to the operator. Terms including “generally,” “about,” “substantially,” and the like, as utilized herein, are meant to encompass variations, e.g., manufacturing tolerances, material tolerances, use and environmental tolerances, measurement variations, and/or other variations, up to and including plus or minus 10 percent. Further, any or all of the aspects described herein, to the extent consistent, may be used in conjunction with any or all of the other aspects described herein.

Provided in accordance with aspects of the present disclosure is a tissue resection device including a handpiece and an end effector assembly extending distally from the handpiece. The end effector assembly includes an outer shaft and an inner member. The outer shaft defines a longitudinal axis and includes a window defined therethrough towards a distal end thereof. The inner member is rotationally disposed within the outer shaft and includes a proximal body portion and a distal cutting portion. The proximal body portion is coaxially disposed on the longitudinal axis and rotatable thereabout. The distal cutting portion includes an offset portion that is radially offset from the longitudinal axis at any variety of fixed or variable angles and is configured to orbit about the longitudinal axis. At least a portion of the distal cutting portion is a thermal cutting element configured to heat in response to application of electrical energy thereto. Rotation of the inner member together with heating of the thermal cutting element facilitate cutting tissue extending through the window and into the outer shaft. In aspects, multiple thermal cutting elements on the same or different rotating axes (rotating in similar or different directions).

In an aspect of the present disclosure, the handpiece includes a motor and an output coupler connected to the motor and the end effector assembly includes an input coupler connected to the inner member. The input coupler is configured to engage the output coupler to enable the motor to drive rotation of the inner member. In such aspects, the end effector assembly may further be configured to releasably engage the handpiece to thereby releasably couple the output and input couplers with one another.

In another aspect of the present disclosure, the handpiece is adapted to connect to a vacuum source to establish suction through the outer shaft, thereby facilitating drawing tissue through the window and into the outer shaft for cutting with the inner member.

In still another aspect of the present disclosure, the offset portion of the distal cutting portion of the cutting member defines a U-shaped configuration.

In yet another aspect of the present disclosure, the thermal cutting element includes a substrate treated with Plasma Electrolytic Oxidation (PEO). Alternatively, the thermal cutting element includes a core coated with a ferromagnetic material. Further, the thermal cutting element may be resistively heated.

In still yet another aspect of the present disclosure, the distal cutting portion of the inner member is rotatably supported within a hub on an interior surface of the outer shaft. Alternatively, the distal cutting portion is “floating” and support is provided at a proximal end portion.

In another aspect of the present disclosure, the handpiece further includes an outflow path defined therethrough and disposed in communication with an interior of the outer shaft.

In yet another aspect of the present disclosure, the distal cutting portion of the inner member at least partially overlaps the window.

In another aspect of the present disclosure, the distal cutting portion of the inner member includes a wire or otherwise formed or shaped electrical conductor.

### BRIEF DESCRIPTION OF THE DRAWINGS

Various aspects and features of the present disclosure are described hereinbelow with reference to the drawings wherein like numerals designate identical or corresponding elements in each of the several views.

FIG. 1 is a perspective view of a tissue resecting system configured for use in hysteroscopic and other surgical procedures;

FIG. 2A is a longitudinal, cross-sectional view of a distal end portion of a tissue resecting device of the tissue resecting system of FIG. 1, wherein the cutting element is disposed in a first position;

FIG. 2B is a longitudinal, cross-sectional view of the distal end portion of the tissue resecting device of FIG. 2A, wherein the cutting element is disposed in a second position;

FIG. 3A is a longitudinal, cross-sectional view of a portion of the tissue resecting device of the tissue resecting system of FIG. 1, wherein the end effector assembly is disengaged from the handpiece; and

FIG. 3B is a longitudinal, cross-sectional view of the portion of the tissue resecting device illustrated in FIG. 3A, wherein the end effector assembly is engaged with the handpiece.

### DETAILED DESCRIPTION

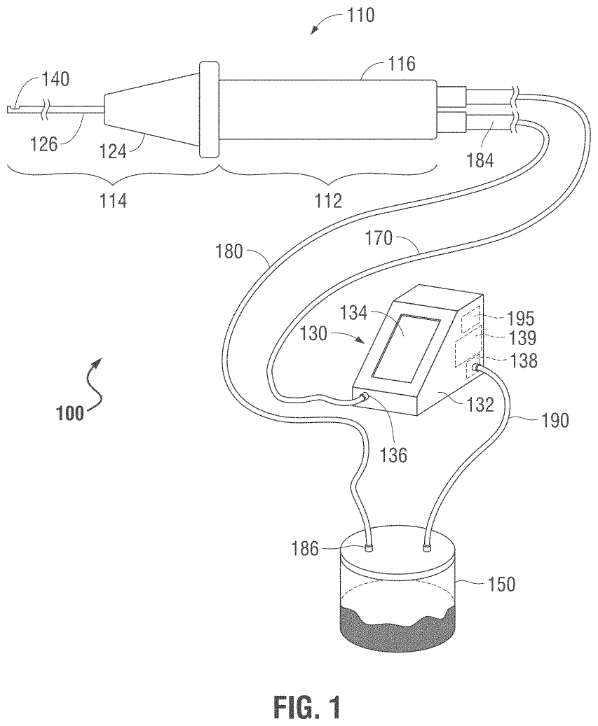

Referring to FIG. 1, a surgical system provided in accordance with aspects of the present disclosure is shown generally identified by reference numeral 100. Surgical system 100 includes a surgical device 110, a control console 130, and a collection vessel 150. Surgical system 100 further includes a cable 170, outflow tubing 180, and vacuum tubing 190. Surgical system 100 may further include an endoscope (not shown), e.g., a hysteroscope, defining a working channel for inserting of surgical device 110 therethrough, and adapted to connect to inflow tubing (not shown) to supply fluid to an internal surgical site and/or additional outflow tubing (not shown) to return fluid to collection vessel 150. Alternatively, surgical system 100 may be used in other surgical procedures, e.g., itself or inserted through a laparoscope (not shown), or in open surgical procedures.

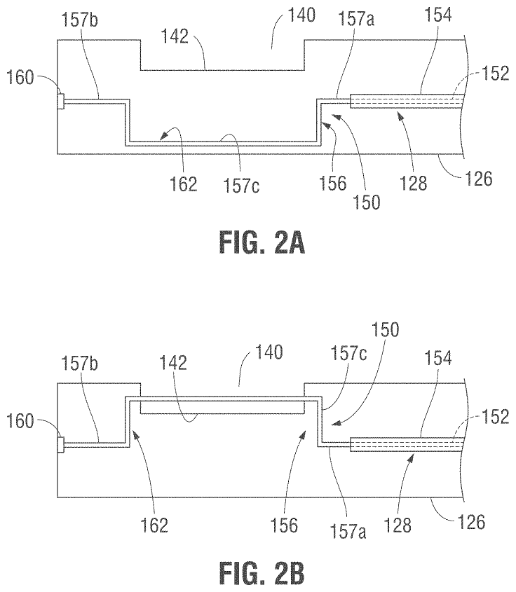

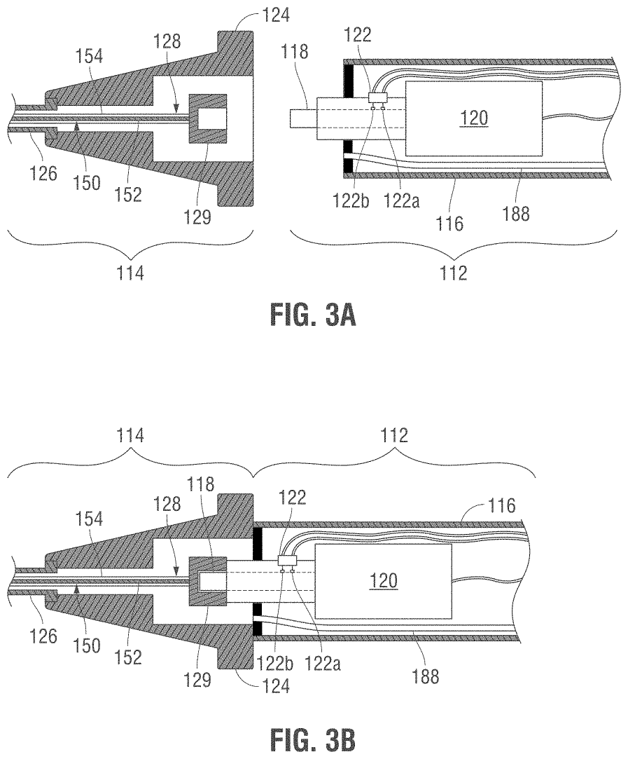

Referring also to FIGS. 2A-3B, surgical device 110 includes a handpiece 112 that may be configured as a reusable component and an end effector assembly 114 that may be configured as a single-use, disposable component. Handpiece 112 includes a housing 116 to facilitate grasping and manipulation of surgical device 110 by a user. Handpiece 112 further includes an output coupler 118 configured to operably engage end effector assembly 114, a motor 120 disposed within housing 116 and operably coupled to output coupler 118 to drive output coupler 118 and, thus, drive end effector assembly 114, and an electrical connection assembly 122 configured to electrically couple to cutting element 150 of end effector assembly 114 to enable energization of cutting element 150 of end effector assembly 114, as detailed below. Electrical connection assembly 122 may include a pair of electrical contacts 122a, 122b that are electrically isolated from one another and adapted to connect to first and second portions of cutting element 150, e.g., to facilitate connection of different potentials of electrical energy to the first and second portions, respectively, of cutting element 150. Electrical connection assembly 122 may be a rotational contact assembly to enable electrical communication via the rotating output coupler 118 or may define any other suitable configuration. In embodiments, rather than electrical contacts 122a, 122b connecting to different portions of cutting element 150, one of the electrical contacts 122a, 122b may connect to a first portion of cutting element 150 and the other of the electrical contacts 122a, 122b may connect to outer shaft 126 such that which is ultimately electrically coupled to a second portion of cutting element 150 via the receipt of a distal end portion of distal segment 157b of cutting element 150 within hub 160 which is formed on or connected with outer shaft 126.

Cable 170 electrically couples handpiece 112 and control console 130 with one another and, more specifically: electrically couples control console 130 with motor 120 to power and control operation of motor 120; electrically couples control console 130 with a storage device(s), e.g., a microchip(s) (not explicitly shown), associated with handpiece 112 and/or end effector assembly 114 to enable communication of, for example, identification, setting, and control information therebetween; and electrically couples cutting element 150 of end effector assembly 114 with a generator 195 disposed within control console 130 via electrical connection assembly 122 to enable selective energization of cutting element 150 of end effector assembly 114 and control of the energization thereof. In embodiments, cable 170 is fixedly attached to handpiece 112 and releasably couplable with control console 130, although other configurations are also contemplated.

End effector assembly 114 includes a proximal hub 124 configured to releasably engage housing 116 of handpiece 112 to releasably mechanically engage end effector assembly 114 with handpiece 112. End effector assembly 114 further includes an outer shaft 126 extending distally from proximal hub 124 and an inner shaft 128 extending through outer shaft 126. A proximal end portion of inner shaft 128 extends into proximal hub 124 wherein an input coupler 129 is engaged with inner shaft 128. Input coupler 129 is configured to operably couple to output coupler 118 of handpiece 112 when proximal hub 124 is engaged with housing 116 such that, when motor 120 is activated to drive rotation of output coupler 118, input coupler 129 is driven to rotate in a corresponding manner to thereby rotate inner shaft 128 within and relative to outer shaft 126. Output and input couplers 118, 129, respectively, may be directly coupled to achieve an output to input ratio of 1:1, e.g., wherein the rotation output from output coupler 118 equals the rotation input to input coupler 129, or may be amplified or attenuated, e.g., using suitable gearing (not shown), to achieve an output to input ratio of greater than or less than 1:1. Inner shaft 128 is coaxially disposed on a longitudinal axis of outer shaft 126 and is configured to rotate about the longitudinal axis.

Outer shaft 126, as noted above, extends distally from proximal hub 124 and, in embodiments, is stationary relative to proximal hub 124, although other configurations are also contemplated. As illustrated in FIGS. 2A and 2B, outer shaft 126 may define a window 140 through a side wall thereof towards a distal end thereof to provide access to cutting element 150 which is rotatably disposed within outer shaft 126. At least a portion of the edge 142 of outer shaft 126 that extends about and defines window 140 may be a sharpened cutting edge and/or a dull edge. As an alternative or in addition to a side-facing window, outer shaft 126 may define an at least partially open distal end for receipt and resection of tissue therethrough. Further, in embodiments, outer shaft 126 and inner shaft 128 may be flexible, e.g., steerable, malleable, or otherwise configured to define various non-linear shapes to better position the distal end portions thereof for resecting tissue.

Continuing with reference to FIGS. 2A and 2B, cutting element 150 may form part of, the entirety of, or may be distinct from and coupled to inner shaft 128. More specifically, in embodiments, as illustrated, cutting element 150 includes a body portion 152 that, together with an insulative sleeve 154 disposed thereabout, forms inner shaft 128. Inner shaft 128, including body portion 152 of cutting element 150 thereof, defines a generally linear configuration and extends coaxially within outer shaft 126. Cutting element 150 further includes a distal cutting portion 156 that extends distally from insulative sleeve 154. Alternatively, cutting element 150 may constitute distal cutting portion 156 and be coupled to a distal end portion of inner shaft 128. In either configuration, inner shaft 128 may electrically couple distal cutting portion 156 of cutting element 150 with electrical connection assembly 122 when end effector assembly 114 is engaged with handpiece 112 (see also FIGS. 3A and 3B).

Distal cutting portion 156 of cutting element 150 includes a proximal segment 157a, a distal segment 157b, and a tissue cutting segment 157c disposed between and connecting proximal and distal segments 157a, 157b, respectively. A proximal end portion of proximal segment 157a is formed with or coupled to inner shaft 128 and proximal segment 157a extends distally therefrom. A distal end portion of distal segment 157b is rotatably received within a hub 160 disposed on an interior surface of outer shaft 126, e.g., an interior distal surface of outer shaft 126, to translationally fix distal segment 157b relative to outer shaft 126 while allowing rotation thereof. Distal segment 157b extends proximally from hub 160. Proximal and distal segments 157a, 157b, respectively, define generally linear configurations and extend coaxially within outer shaft 126. Thus, upon rotation of inner shaft 128, proximal and distal segments 157a, 157b, respectively, likewise rotate about the longitudinal axis of outer shaft 126.

Tissue cutting segment 157c of distal cutting portion 156 of cutting element 150 includes an offset portion 162 that is at least partially offset from the longitudinal axis of outer shaft 126 such that, upon rotation of inner shaft 128, the offset portion 162 orbits about (in radially-spaced relation relative to) the longitudinal axis. Offset portion 162 is positioned to at least partially overlap with window 140 of outer shaft 126 and may define a U-shaped configuration (as shown), a C-shaped configuration, an S-shaped configuration, or any other suitable angled and/or arcuate configuration that includes a portion that extends from and returns to the longitudinal axis. In embodiments, plural distal cutting portions 156 are provided, e.g., offset relative to one another. For example, four distal cutting portions 156 may be provided offset 90 degrees relative to one another. Other configurations are also contemplated.

Cutting element 150 may be any suitable thermal cutting element such as, for example, an aluminum (or other suitable) substrate that is Plasma Electrolytic Oxidation (PEO)-treated at least along a portion of tissue cutting segment 157c such that when an AC voltage is applied, the PEO-treated portion thereof is heated for thermally cutting tissue. As another example, tissue cutting segment 157c of cutting element 150 may be configured as a ferromagnetic (FM) element including a core, e.g., copper, and a ferromagnetic material coated on the core such that when an AC voltage is applied, the FM element is heated up to the Curie point (thus providing automatic, Curie-point temperature control) for thermally cutting tissue. Other suitable cutting element configurations are also contemplated. Further still, tissue cutting segment 157c of cutting element 150 may be a resistively heated wire. In any of the above configurations, a portion of tissue cutting segment 157c may be configured to be energized, the entire tissue cutting segment 157c may be configured to be energized, and/or additional or alternative portions of distal cutting portion 156 of cutting element may be configured to be energized. Cable 170 (FIG. 1) or a separate cable may include a coaxial cable for conducting high-frequency energy for energizing cutting element 150 in embodiments where cutting element 150 is an FM element.

Referring also to FIG. 1, motor 120 of handpiece 112, as noted above, is activated to drive rotation of inner shaft 128 relative to outer shaft 126. Control console 130, coupled to motor 120 via cable 170, enables selective powering and controlling of motor 120 and, thus, selective rotation of inner shaft 128 (and tissue cutting segment 157c) relative to outer shaft 126 to resect tissue extending into window 140 of outer shaft 126. Control console 130 and, more specifically, generator 195 thereof, may be configured to supply energy to activate tissue cutting segment 157c (and/or other portions of) of cutting element 150 simultaneously with the activation of rotation of inner shaft 128, in overlapping relation therewith, independently thereof, or in any other suitable manner such that, in at least some modes and/or portions of operation, energization and the resultant thermal heating of tissue cutting segment 157c (and/or other portions of) of cutting element 150 may facilitate cutting tissue as tissue cutting segment 157c is rotated within and relative to outer shaft 126 adjacent window 140 thereof.

Outflow tubing 180 includes a distal end 184 configured to couple to handpiece 112 and a proximal end 186 configured to couple to collection vessel 150. More specifically, handpiece 112 defines an internal conduit 188 (FIGS. 3A-3B) that couples distal end 184 of outflow tubing 180 with the interior of outer shaft 126 (FIGS. 3A-3B) in fluid communication therewith such that fluid, cut tissue, and debris drawn into outer shaft 126 may be suctioned, under vacuum, e.g., from a vacuum pump 139 of control console 130, through end effector assembly 114, handpiece 112, and outflow tubing 180, to collection vessel 150. In embodiments, irrigation may also be provided via inflow tubing (not shown) and through outer shaft 126 (FIGS. 3A and 3B).

With reference to FIG. 1, collection vessel 150, as noted above, is coupled to proximal end 186 of outflow tubing 180 to receive the fluid, cut tissue, and debris suctioned through end effector assembly 114 and outflow tubing 180. Vacuum tubing 190 is coupled between collection vessel 150 and vacuum pump 139 of control console 130, such that, upon activation of vacuum pump 139, negative pressure is established through collection vessel 150, outflow tubing 180, and the interior of outer shaft 126 of end effector assembly 114 to draw tissue through window 140 of outer shaft 126 to facilitate cutting thereof, and to draw fluids, cut tissue, and debris proximally through outer shaft 126, handpiece 112 and outflow tubing 180 into collection vessel 150.

As an alternative or in addition to establishing suction through interior of outer shaft 126 via vacuum pump 139, flow can be created via a pressure differential between the interior and exterior of outer shaft 126 via by pumping fluid into the uterus (or other body cavity) to establish a positive intrauterine pressure. This may cause tissue, fluid, and/or debris to pass through window 140 and into outer shaft 126. Further, this enables tissue resection upon pressing the distal end portion of outer shaft 126 into contact with tissue, e.g., against the uterine wall or polyp, and subsequent removal of the tissue through window 140 and into outer shaft 126 such that resected tissue, e.g., diseased tissue such as cancerous cells, are removed from the uterus.

Control console 130 generally includes an outer housing 132, a touch-screen display 134 accessible from the exterior of outer housing 132, a cable port 136 configured to receive cable 170, a vacuum tubing port 138 configured to receive vacuum tubing 190, a vacuum pump 139 disposed within outer housing 132 and operably coupled with vacuum port 138, and a generator 195 electrically coupled with cable port 136. Outer housing 132 further houses internal electronics (not shown) of control console 130. Control console 130 may be configured to connect to a mains power supply (not shown) for powering control console 130. Further, control console 130 may be configured to receive user input, e.g., use information, setting selections, etc., via touch-screen display 134 or a peripheral input device (not shown) coupled to control console 130. Operational input, e.g., ON/OFF signals, power level settings (HI power vs. LO power), thermal cutting mode and/or temperature settings, etc., may likewise be input or selected via touch-screen display 134 or a peripheral input device (not shown) such as, for example, a footswitch (not shown), a handswitch (not shown) disposed on handpiece 112, etc.

Referring also to FIGS. 2A-3B, in use, upon an activation input provided to control console 130, control console 130 powers and controls motor 120 of handpiece 112 to, in turn, drive inner shaft 128 of end effector assembly 114 to rotate, thereby rotating tissue cutting segment 157c. Alternatively, inner shaft 128 may be stationary and outer shaft 126 may be configured to rotate thereabout. Ahead of the driving of inner shaft 128, simultaneously therewith, or delayed thereafter, vacuum pump 139 of control console 130 is activated to suction fluid, tissue, and debris through window 140, outer shaft 126, handpiece 112, outflow tubing 180, and into collection vessel 150. The suction provided from vacuum pump 139 also facilitates suctioning of tissue through window 140 of outer shaft 126 such that the rotating tissue cutting segment 157c can resect the tissue extending through window 140 into outer shaft 126. Alternatively or additionally, a pressure differential resulting from a positive intrauterine (or intracavity) pressure may allow for drawing of tissue through window 140 of outer shaft 126. Further, ahead of the driving of inner shaft 128, simultaneously therewith, or delayed thereafter, generator 195 of control console 130 is activated to energize tissue cutting segment 157c such that tissue cutting segment 157c is heated to enable thermal tissue cutting, thus facilitating the resection and removal of tissue through device 110.

In embodiments, additional tools and/or features may be incorporated into device 110 such as, for example, a camera, grasper, etc.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as examples of particular embodiments. Those skilled in the art will envision other modifications within the scope and spirit of the claims appended hereto.

Although the foregoing disclosure has been described in some detail by way of illustration and example, for purposes of clarity or understanding, it will be obvious that certain changes and modifications may be practiced within the scope of the appended claims.

What is claimed is:

1. A tissue resection device, comprising:
a handpiece; and
an end effector assembly extending distally from the handpiece, the end effector assembly including:
an outer shaft defining a longitudinal axis and including a window defined therethrough towards a distal end thereof; and
an inner member rotationally disposed within the outer shaft, the inner member including a proximal body portion and a distal cutting portion, the proximal body portion coaxially disposed on the longitudinal axis and rotatable thereabout, the distal cutting portion including an offset portion that is radially offset from the longitudinal axis and is configured to orbit about the longitudinal axis, wherein at least a portion of the distal cutting portion is a thermal cutting element configured to heat in response to application of electrical energy thereto, wherein rotation of the inner member together with heating of the thermal cutting element facilitate cutting tissue extending through the window and into the outer shaft.

2. The tissue resection device according to claim 1, wherein the handpiece includes a motor and an output coupler connected to the motor, wherein the end effector assembly includes an input coupler connected to the inner member, and wherein the input coupler is configured to engage the output coupler to enable the motor to drive rotation of the inner member.

3. The tissue resection device according to claim 2, wherein the end effector assembly is configured to releasably engage the handpiece to thereby releasably couple the output and input couplers with one another.

4. The tissue resection device according to claim 1, wherein the handpiece is adapted to connect to a vacuum source to establish suction through the outer shaft, thereby facilitating drawing tissue through the window and into the outer shaft for cutting with the inner member.

5. The tissue resection device according to claim 1, wherein the offset portion of the distal cutting portion of the cutting member defines a U-shaped configuration.

6. The tissue resection device according to claim 1, wherein the thermal cutting element includes a substrate treated with Plasma Electrolytic Oxidation (PEO).

7. The tissue resection device according to claim 1, wherein the thermal cutting element includes a core coated with a ferromagnetic material.

8. The tissue resection device according to claim 1, wherein the thermal cutting element is resistively heated.

9. The tissue resection device according to claim 1, wherein the distal cutting portion of the inner member is rotatably supported within a hub on an interior surface of the outer shaft.

10. The tissue resection device according to claim 1, wherein the handpiece further includes an outflow path defined therethrough and disposed in communication with an interior of the outer shaft.

11. The tissue resection device according to claim 1, wherein the distal cutting portion of the inner member at least partially overlaps the window.

12. The tissue resection device according to claim 1, wherein the distal cutting portion of the inner member includes a wire.
